(19) United States
(12) Patent Application Publication
(10) Pub. No.: US 2026/0053918 A1
(43) Pub. Date: Feb. 26, 2026

(54) Use of Triplex CMV Vaccine in CAR T Cell Therapy

(71) Applicant: City of Hope, Duarte, CA (US)

(72) Inventors: Don J. Diamond, Duarte, CA (US); Xiuli Wang, Temple City, CA (US); Stephen J. Forman, Duarte, CA (US)

(21) Appl. No.: 19/370,102

(22) Filed: Oct. 27, 2025

Related U.S. Application Data
(63) Continuation of application No. 18/354,849, filed on Jul. 19, 2023, now Pat. No. 12,478,672.
Application No. 18/354,849 is a continuation of application No. 17/572,496, filed on Jan. 10, 2022, now abandoned.
Application No. 17/572,496 is a continuation of application No. 17/006,758, filed on Aug. 28, 2020, now abandoned.
Application No. 17/006,758 is a continuation of application No. 16/343,701, now abandoned.
(60) Provisional application No. 62/410,372, filed on Oct. 19, 2016.

(51) Int. Cl. A61K 39/395; A61K 39/00; A61K 39/12; A61K 39/245; A61K 39/285; A61K 40/11; A61K 40/31; A61K 40/42; A61K 40/46; A61P 31/20; A61P 35/00; C07K 14/00; C07K 14/005; C07K 14/705; C07K 14/725; C07K 14/73; C07K 16/28; C12N 5/0783; C12N 15/86

(52) U.S. Cl. CPC A61K 39/39558; A61K 39/12; A61K 39/245; A61K 39/285; A61K 40/11; A61K 40/31; A61K 40/4211; A61K 40/46; A61P 31/20; A61P 35/00; C07K 14/00; C07K 14/005; C07K 14/7051; C07K 14/70514; C07K 14/70517; C07K 14/70578; C07K 16/2803; C12N 5/0636; C12N 15/86; A61K 2039/5256; A61K 2039/585; C07K 2319/03; C07K 2319/33; C12N 2710/16122; C12N 2710/16134; C12N 2710/24134; C12N 2710/24143

(57) ABSTRACT

A method for treating a patient comprising: (a) providing a composition comprising a population of T cells expressing both a chimeric antigen receptor (CAR) and a T cell receptor specific for a cytomegalovirus (CMV) antigen; (b) administering the composition to the patient; and (c) administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient is described.

### CROSS REFERENCE TO RELATED APPLICATIONS

This application is a continuation of U.S. application Ser. No. 18/354,849, filed on Jul. 19, 2023, which is a continuation of U.S. application Ser. No. 17/572,496, filed on Jan. 10, 2022, which is a continuation of U.S. application Ser. No. 17/006,758, filed on Aug. 28, 2020, which is a continuation of U.S. application Ser. No. 16/343,701, filed on Apr. 19, 2019, which is a National Stage application under 35 U.S.C. § 371 of International Application No. PCT/US2017/057433, filed on Oct. 19, 2017, which claims the benefit of U.S. Provisional Application No. 62/410,372, filed on Oct. 19, 2016. The disclosures of the prior applications are incorporated by reference in their entirety.

### STATEMENT OF FEDERALLY SPONSORED RESEARCH

This invention was made with government support under CA107399, CA030206, and CA077544, awarded by the National Institutes of Health. The government has certain rights in the invention.

### SEQUENCE LISTING

This application contains a Sequence Listing that has been submitted electronically as an XML file named “40056-0030005_SL_ST26.XML”. The XML file, created on Oct. 23, 2025, is 48,263 bytes in size. The material in the XML file is hereby incorporated by reference in its entirety.

### BACKGROUND

Tumor-specific T cell based immunotherapies, including therapies employing engineered T cells, have been investigated for anti-tumor treatment. In some cases, the T cells used in such therapies do not remain active in vivo for a long enough period.

Over 70,000 new cases of non-Hodgkin lymphoma (NHL) are diagnosed each year in the United States with about 20,000 deaths due to NHL each year, representing the 5th leading cause of cancer deaths. The majority of these patients have widespread disease at the time of diagnosis and over two-thirds will suffer a recurrence after remission induction with cytotoxic chemotherapy and rituximab. Efforts to improve the survival of patients with recurrent lymphoma have focused mainly on the use of autologous hematopoietic cell transplant (HCT), which is curative in approximately half of good-risk patients, but confers a less than 15% 5-year event-free survival in patients with poor prognostic features. Allogeneic HCT provides a tumor-free stem cell graft, cells that have not been damaged by prior chemotherapy and the opportunity for graft-versus-lymphoma (GVL) effect and has been increasingly applied in patients with relapsed NHL. Although relapse rates are improved over autologous HCT, allogeneic HCT is associated with both significant risks of transplant-related complications and also disease recurrence. Thus, there is an important need for the development of new therapies that can consolidate the tumor cytoreduction achieved with autologous or allogeneic HCT by eradicating the limited number of tumor cells surviving after autologous myeloablative and reduced intensity allogeneic conditioning. A Phase I clinical trial using ex vivo-expanded autologous central memory-enriched T cells (TCM) transduced with lentivirus expressing CD19-specific CAR has demonstrated the data safety and feasibility of CD19 CAR T cell therapy after HCT Wang et al., Blood 127:2980, 2016).

### SUMMARY

Described herein is the use of a cytomegalovirus (CMV) Triplex Vaccine in combination with engineered T cells that both recognize a CMV antigen and express a chimeric antigen receptor target to an antigen expressed on normal B cells as well as on cancerous cells (CMV/CAR T cells) to treat a variety of cancers. The methods entail administering CMV/CAR T cells which recognize a tumor antigen (e.g., CD19) in addition to a CMV antigen to a patient. Subsequent to administration of the CMV/CAR T cells, a CMV Triplex Vaccine is administered to the patient. The vaccine can promote proliferation of the CMV/CAR T cells and enhance their anti-tumor activity. Thus, the methods can improve T cell resistance and provide a means by which to re-stimulate CAR T cells after relapse. In addition, the methods can provide more reliable engraftment and persistence in a low target-antigen setting (e.g., post-myeloablative HCT) with re-expansion of CAR T cells by CMV vaccine administration. The methods described herein also permit in vivo expansion of CMV-specific CAR T cells, instead of or in addition to ex vivo expansion, avoiding excessive T cell exhaustion that results in some cases from ex vivo manufacturing.

The CMV/CAR T cells can be prepared by a method comprising: (a) providing PBMC from a cytomegalovirus (CMV)-seropositive human donor; (b) exposing the PBMC to at least one CMV antigen (e.g., pp65 or a mixture of IE1/IE2 overlapping peptides); (c) treating the exposed cells to produce a population of cells enriched for stimulated cells specific for CMV (e.g., treating them to create a population of cells that is enriched for stimulated cells specific for CMV relative to the untreated population of cells); (d) transducing at least a portion of the enriched population of cells with a vector (e.g., a lentiviral vector) expressing a CAR, thereby preparing T cells specific for CMV and expressing a CAR. In some cases, a CMV vaccine, for example, the CMV Triplex Vaccine, can be administered to the donor prior to harvest of the PBMC in order to increase the frequency of CMV-positive T cells.

The CMV Triplex Vaccine is a recombinant MVA expressing a fusion protein of two CMV antigens, IE1-exon4 and IE2-exon5 and CMV antigen pp65. The sequence encoding the fusion protein is inserted in the MVA deletion-II locus and the sequence encoding the CMV pp65 antigen is inserted into the MVA deletion-III locus. The CMV Triplex Vaccine is described in greater detail in U.S. Pat. No. 8,580,276, hereby incorporated by reference.

The methods described herein include: a method for treating a patient comprising: (a) providing a composition comprising a population of T cells expressing both a chimeric antigen receptor (CAR) and a T cell receptor specific for a cytomegalovirus (CMV) antigen; (b) administering the composition to the patient; and (c) administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient.

Described herein is a method for treating a patient comprising: (a) providing a composition comprising a population of T cells expressing both a chimeric antigen receptor (CAR) and a T cell receptor specific for a cytomegalovirus (CMV) antigen; (b) administering the composition to the patient; and (c) administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient.

In various embodiments: the step of administering a viral vector to the patient comprises administering recombinant MVA virus; expression of (i) CMV pp65 and (ii) the fusion protein comprising exon 4 of CMV protein IE1 (e4) and exon 5 of CMV protein IE2 (e5) is under the control of mH5 promoter; the patient is immunocompromised; the patient is immunocompetent; the patient is CMV-seronegative prior to treatment; the patient is CMV-seropositive prior to treatment; the patient received hematopoietic stem cells (HSC) from a CMV-positive or CMV-negative donor prior to administering the comprising a population of T cells expressing both a chimeric antigen receptor (CAR) and a T cell receptor specific for a cytomegalovirus (CMV) antigen; and the CAR is targeted to CD19; administration of the viral vector occurs at least 5 days after treatment with the composition comprising a population of T cells; the viral vector is administered to the patient both prior to and subsequent to the administration of the composition comprising a population of T cells; the viral vector is administered to the hematopoietic stem cell donor prior to harvesting the stem cells; the viral vector is administered to the patient only prior to the administration of the composition comprising a population of T cells; the viral vector is administered to the patient only subsequent to the administration of the composition comprising a population of T cells; the viral vector is administered to the patient at least four times; the patient is suffering from non-Hodgkin's Lymphoma.

The CAR is selective can be selective for any antigen, for example: CD19, CS1, CD123, 5T4, 8H9, αvβ6 integrin, alphafetoprotein (AFP), B7-H6, CA-125 carbonic anhydrase 9 (CA9), CD19, CD20, CD22, CD30, CD33, CD38, CD44, CD44v6, CD44v7/8, CD52, CD123, CD171, carcinoembryonic antigen (CEA), EGFrvIII, epithelial glycoprotein-2 (EGP-2), epithelial glycoprotein-40 (EGP-40), ErbB1/EGFR, ErbB2/HER2/neu/EGFR2, ErbB3, ErbB4, epithelial tumor antigen (ETA), FBP, fetal acetylcholine receptor (AchR), folate receptor-α, G250/CAIX, ganglioside 2 (GD2), ganglioside 3 (GD3), HLA-A1, HLA-A2, high molecular weight melanoma-associated antigen (IVMW-MAA), IL-13 receptor α2, KDR, k-light chain, Lewis Y (LeY), L1 cell adhesion molecule, melanoma-associated antigen (MAGE-A1), mesothelin, Murine CMV infected cella, mucin-1 (MUC1), mucin-16 (MUC16), natural killer group 2 member D (NKG2D) ligands, nerve cell adhesion molecule (NCAM), NY-ESO-1, Oncofetal antigen (h5T4), prostate stem cell antigen (PSCA), prostate-specific membrane antigen (PSMA), receptor-tyrosine kinase-like orphan receptor 1 (ROR1), TAA targeted by mAb IgE, tumor-associated glycoprotein-72 (TAG-72), tyrosinase, and vascular endothelial growth factor (VEGF) receptors.

In certain embodiments: the CAR is selective for an antigen selected from: CD19, CD123, CS1, BCMA, CD44v6, CD33, CD22, IL-13α2, PSA, HER-2, EGFRv3, CEA, and C7R; the CAR comprises: a scFv selective for the selected non-CMV antigen; a hinge/linker region; a transmembrane domain; a co-signaling domain; and CD3ζ signaling domain; the co-signaling domain is selected from a CD28 co-signaling domain and a 4-IBB co-signaling domain; transmembrane domain is selected from a CD28 transmembrane domain and a CD4 transmembrane domain.

In various embodiments of the treatment method: the population of human T cells is autologous to the patient; the population of human T cells is allogeneic to the patient; the method reduces the risk of CMV infection; the method reduces CMV viremia and/or disease; the patient was CMV-immune prior to treatment and the method reduces the risk of CMV infection; the patient was not CMV-immune prior to treatment and the method reduces CMV viremia or disease.

In some embodiments, the step of providing a population of T cells expressing a CAR and a T cell receptor specific for a CMV antigen comprises: (a) providing PBMC or a T cell subpopulation from a CMV-seropositive human donor; (b) exposing the PBMC or the T cell subpopulation to at least one CMV antigen; (c) treating the exposed cells to produce a population of cells enriched for stimulated cells specific for CMV; (d) transducing at least a portion of the enriched population of cells with a vector expressing a CAR. In some embodiments, the step of treating the exposed cells to produce a population of cells enriched for stimulated cells specific for CMV comprises treating the stimulated cells to produce a population of cells enriched for cells expressing an activation marker.

In some embodiments, the step of providing a population of T cell expressing a CAR and a T cell receptor specific for a CMV antigen comprises: (a) administering a viral vector encoding: (i) CMV pp65 and (ii) a fusion protein comprising exon 4 of CMV protein IE1 (e4) and exon 5 of CMV protein IE2 (e5) to a human donor to convert a CMV-seronegative human donor to one containing T cells responsive to CMV antigens pp65, IE1 and IE2; (b) obtaining PBMC from the CMV-seropositive human donor; (c) exposing the PBMC to at least one CMV antigen; (d) treating the exposed cells to produce a population of cells enriched for stimulated cells specific for CMV; (e) transducing at least a portion of the enriched population of cells with a vector expressing a CAR, thereby providing a population of T cell expressing a CAR and a T cell receptor specific for a CMV antigen.

In some embodiments, the step of providing a population of T cell expressing a CAR and a T cell receptor specific for a CMV antigen comprises: (a) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

In the case of patients who have received HSC transplant, in some embodiments, the viral vector is administered to the patient or the hematopoietic stem cell transplant donor at least twice subsequent to the administration of the composition comprising a population of T cells, the hematopoietic stem cells were autologous to the patient; and the hematopoietic stem cells were allogenic to the patient.

Also described is a method for preparing T cells expressing a CAR and a T cell receptor specific for a CMV antigen, the method comprising: (a)) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

Also described is a method for preparing T cells expressing a CAR and a T cell receptor specific for a CMV antigen, the method comprising: a)) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

In various embodiments of the of the methods for producing T cells, the method further comprises expanding the population of T cell expressing a CAR and a T cell receptor specific for a CMV antigen.

In various embodiments of the of the methods for producing T cells: the activation marker is IFN-γ or other activation marker such as CD137, CD107 or other cytokines; the CMV antigen is pp65 protein or an antigenic portion thereof, the CMV antigen comprises two or more different antigenic CMV pp65 peptides; the step of transducing the enriched population of cells does not comprise CD3 stimulation; the step of transducing the enriched population of cells does not comprise CD28 stimulation; the step of transducing the enriched population of cells does not comprise CD28 stimulation or CD3 stimulation; the step of transducing the enriched population of cells does not comprise exposing the cells to an anti-CD28 antibody or an anti-CD3 antibody; the enriched population of cells is at least 40% IFN-γ positive, at least 20% CD8 positive, and at least 20% CD4 positive; the enriched population of cells are cultured for fewer than 10 days prior to the step of transducing the enriched population of cells with a vector encoding a CAR; the method further comprises expanding the CMV specific T cells expressing a CAR cells by exposing them to an antigen that binds to the CAR; the step of expanding the CMV-specific T cells expressing a CAR comprises exposing the cells to T cells expressing the antigen that binds the CAR; and the expansion takes place is the presence of at least one exogenously added interleukin.

In some cases, the method includes a step of preparing T cells specific for cytomegalovirus (CMV) and expressing a chimeric antigen receptor (CAR), the method comprising: (a) providing T cells (e.g., PBMC) from a cytomegalovirus CMV seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby preparing T cells specific for CMV and expressing a CAR. In various cases: the step of treating the exposed cells (e.g., using a selection step) to produce a population of cells enriched for stimulated cells specific for CMV comprises treating the stimulated cells to produce a population of cells enriched for cells expressing an activation marker (e.g., IFN-γ of IL-13); the PBMC are cultured for less than 5 days (less than 4, 3, 2, 1 days) prior to exposure to the CMV antigen; the cells are exposed to the CMV antigen for fewer than 3 days (fewer than 48 hrs, 36 hrs, 24 hrs) the CMV antigen is pp65 protein or an antigenic portion thereof, the CMV antigen comprises two or more different antigenic CMV pp65 peptides; the step of transducing the enriched population of cells does not comprise CD3 stimulation; the step of transducing the enriched population of cells does not comprise CD28 stimulation; the step of transducing the enriched population of cells does not comprise CD3 stimulation or CD28 stimulation; the enriched population of cells is at least 40% (e.g., 50%, 60%, 70%) IFN-γ positive, at least 20% (e.g., 25%, 30%, 35%) CD8 positive, and at least 20% (e.g., 25%, 30%, 35%) CD4 positive; the enriched population of cells are cultured for fewer than 10 (fewer than 9, 8, 7, 5, 3, 2) days prior to the step of transducing the enriched population of cells with a vector encoding a CAR. In some cases, the T cells are from a CMV positive donor and are exposed to a CMV antigen such as CMV pp65 or a mixture of CMV protein peptides (for example 10-20 amino acid peptides that are fragments of pp65) in the presence of IL-2 to create a population of stimulated cells. In some cases, the population of stimulated cells is treated to prepare a population of cells that express IFN-7. In some cases, the CMV/CAR T cells do not recognize an antigen from a second virus. For example, they do not recognize an Epstein-Barr virus antigen or an influenza virus antigen or an Adenovirus antigen.

In some cases, the method further comprises expanding the CMV specific T cells expressing a CAR (CMV/CAR T cells) by exposing them an antigen that binds to the CAR.

In some cases, the CMV/CAR T cells are not expanded ex vivo by exposure to an antigen that binds the CAR, by a CMV antigen or by exposure to exogenously added cytokines.

In some cases, the step of expanding the CMV-specific T cells expressing a CAR comprises exposing the cells to T cells expressing the antigen that bind the CAR (e.g., the expansion takes place is the presence of at least one exogenously added interleukin (e.g., one or both of IL-1 and IL-15) and a T cell expressing the antigen recognized by the CAR.

In various cases: the CAR is selective for an antigen selected from: CD19, CS1, CD123, 5T4, 8H9, αvβ6 integrin, alphafetoprotein (AFP), B7-H6, CA-125 carbonic anhydrase 9 (CA9), CD19, CD20, CD22, CD30, CD33, CD38, CD44, CD44v6, CD44v7/8, CD52, CD123, CD171, carcinoembryonic antigen (CEA), EGFrvIII, epithelial glycoprotein-2 (EGP-2), epithelial glycoprotein-40 (EGP-40), ErbB1/EGFR, ErbB2/HER2/neu/EGFR2, ErbB3, ErbB4, epithelial tumor antigen (ETA), FBP, fetal acetylcholine receptor (AchR), folate receptor-α, G250/CAIX, ganglioside 2 (GD2), ganglioside 3 (GD3), HLA-A1, HLA-A2, high molecular weight melanoma-associated antigen (HMW-MAA), IL-13 receptor α2, KDR, k-light chain, Lewis Y (LeY), L1 cell adhesion molecule, melanoma-associated antigen (MAGE-A1), mesothelin, Murine CMV infected cella, mucin-1 (MUC1), mucin-16 (MUC16), natural killer group 2 member D (NKG2D) ligands, nerve cell adhesion molecule (NCAM), NY-ESO-1, Oncofetal antigen (h5T4), prostate stem cell antigen (PSCA), prostate-specific membrane antigen (PSMA), receptor-tyrosine kinase-like orphan receptor 1 (ROR1), TAA targeted by mAb IgE, tumor-associated glycoprotein-72 (TAG-72), tyrosinase, and vascular endothelial growth factor (VEGF) receptors.

In some cases, the CAR is selective for an antigen selected from: CD19, CD123, CS1, BCMA, CD44v6, CD33, CD22, IL-13α2, PSA, HER2, EGFRv3, CEA, and C7R.

In some cases: the CAR comprises: a scFv selective for the selected non-CMV antigen; a hinge/linker region; a transmembrane domain; a co-signaling domain; and CD3ζ signaling domain; the chimeric antigen receptor further comprises a spacer sequence located between the co-signaling domain and the CD3ζ signaling domain; the co-signaling domain is selected from a CD28 co-signaling domain and a 4-IBB co-signaling domain; the transmembrane domain is selected from a CD28 transmembrane domain and a CD4 transmembrane domain; the vector expressing the CAR expresses a truncated human EGFR from the same transcript encoding the CAR, wherein the truncated human EGFR lacks a EGF ligand binding domain and lacks a cytoplasmic signaling domain; the spacer sequence comprises or consists of 3-10 consecutive Gly; the hinge/linker region comprises at least 10 amino acids of an IgG constant region or hinge region; the IgG is IgG4; the hinge/linger region comprises an IgG4 CD3 domain; the hinge/linger region comprises an IgG4 Fc domain or a variant thereof, the hinge/linker region comprises or consists of 4-12 amino acids; and hinge/linker region is selected from the group consisting of: the sequence ESKYGPPCPPCPGGGSSGGGSG and the sequence GGGSSGGGSG.

In some cases, the CMV/CAR T cell population is a population in which at least 20% of the cells in the population are CD4+, in which at least 20% of the cells in the population are CD8+, or in which at least 60% of the cells in the population are IFNγ+.

In various cases: the T cells are specific for CMV pp65; and the CAR binds an antigen selected from: CD19, CD123, CS1, BCMA, CD44v6, CD33, CD22, IL-13α2, PSA, HER2, EGFRv3, CEA, and C7R.

Also described is a method of treating a patient suffering from cancer comprising administering a composition comprising CMV/CAR T cells followed by administration of CMV Triplex Vaccine. In various cases: the population of human T cells are autologous to the patient; the population of human T cells are allogenic to the patient; the population of human T cells are autologous to the patient; the method further comprises administering to the patient at least two or at least three doses of a CMV Triplex Vaccine.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

### DESCRIPTION OF DRAWINGS

FIGS. 1A-ID depict the development of clinically feasible platform for derivation of CMV/CAR T cells and the schematic structure of a lentiviral vector expressing a CD19 CAR. (A) CMV-specific T cells from CMV immune HLA A2 donors were selected using IFNγ capture after overnight stimulation with cGMP grade CMVpp65 protein. After selection, the cells were stained with antibodies specific to IFNγ, CD4, and CD8. The frequency of each population is presented after exclusion of dead cells with DAPI. (B) The selected cells were transduced with the second generation CD19CAR with a double mutation in the spacer, 24 hours after the IFNγ capture. 7-10 days later, the transduced cells were stimulated with irradiated CD19 expressing NIH3T3 cells at 10:1 ratio (3T3: T cells) and the stimulation was repeated 7 days post the first stimulation. CAR expression was defined by cetuximab-biotin and streptavidin (SA) APC-Cy7 staining. Percentages of CAR⁺ cells are indicated in each histogram (filled gray), and based on subtraction of that stained with SA-APC-Cy7 alone (black line). (C) Growth of total cell number was determined by Guava Viacount at different time points. (D) Schematic diagram of 10039 nt lentiviral vector encoding a CD19 CAR. Within the 3183 nucleotide long CD19R:CD28:z(CO)-T2A-EGFRt construct, the CD19-specific scFv, IgG4 Fc spacer, theCD28 transmembrane and cytoplasmic signaling domains, three-glycine linker, and CD3z cytoplasmic signaling domains of the CD19R:CD28:z(CO) CAR containing the 2 point mutations, L235E and N297Q, in the CH2 portion of the IgG4 spacer (CD19R(EQ)), as well as the T2A ribosome skip and truncated EGFR sequences are indicated. The human GM-CSF receptor alpha signal sequences that drive surface translocation of the CD19R:CD28:z(CO) CAR and EGFRt are also indicated.

FIGS. 2A-2C depict the results of studies demonstrating that CMV/CAR T cells exhibit specific effector function after engagement with CD19′ and CMVpp65⁺ tumors. (A) 7 days after the second CD19 Ag stimulation, T cells were stained with HLA A2 restricted pp65 tetramer, cetuximab-biotin, anti-CD8 and antibodies specific to central memory T cell surface markers. Percent positive cells are indicated after dead cell exclusion with DAPI, gating based on pp65 tetramer and cetuximab double-positivity, and isotype-matched stained samples. (B) Four-hour ⁵¹Cr release assays were performed using the CMV/CAR T cells and indicated ⁵¹Cr-labeled target cells at different effector:target (E:T) ratios. OKT3-expressing LCLs were used as positive controls, KG1A and U251T as negative controls. CD19⁺ LCL and engineered pp65U251T cells were used as target for CD19 and CMV-specific T cells, respectively. Data from a representative donor is presented. (C) CMV/CAR T cells (105) were activated overnight with 10⁵ LCL-OKT3, LCL, or KG1a in 96-well tissue culture plates and 10⁵ U251T and engineered pp65 expressing U251T cells (pp65U251T) in 24-well tissue culture plates. Supernatants were collected after overnight co-incubation of CMV/CAR T cells and stimulators. Cytokine levels with indicated stimulators (means±SEM of triplicate wells) were determined using cytometric bead array.

FIG. 2D depicts the results of studies examining cytokine levels in the serum of CMV/CAR T cell treated tumor bearing mice. NSG mice were injected i.v. on day 0 with 2.5×106 GFPffluc+ LCL cells. Three days after tumor inoculation, recipient mice were administered i.v. with 2×106 CMV/CAR cells that underwent 2 rounds of CD19 stimulation. Vaccine was given by i.v. injection of peptide (pp65 or MP1) pulsed autologous T cells on day 14. Thirteen days post vaccine, serum of recipient mice was collected and levels of human cytokines were determined by cytometric bead array. Cytokine levels in the serum of untreated mice was used as baseline. Mean and SEMs from triplicates are presented.

FIGS. 3A-3C depicts the mH5-IEfusion-pZWIIA (GUS) plasmid DNA sequence (SEQ ID NO: 29).

FIGS. 4A-4C depicts the mH5-pp65-pLW51(GUS) plasmid DNA sequence (SEQ ID NO: 30).

FIGS. 5A-5C depicts an IE1 antigen and the IE2 antigen fusion protein (SEQ ID NO: 31).

### DETAILED DESCRIPTION

Described below are T cells specific for CMV and CD19. These CMV/CAR T cells were generated using a rapid and efficient method for generating and selecting CMV-specific T cells. The method, which employs IFNγ capture of CMV-specific T cells, consistently and efficiently enriched CMV-specific T cells while preserving the broad spectrum of CMV repertoires. Moreover, the cells remained amenable to gene modification after a brief CMVpp65 stimulation, avoiding the need for CD3/CD28 bead activation prior to transduction. This is significant because CD3/CD28 activation can cause activation-induced cell death (AICD) of CMV-specific T cells. Engineering the bulk IFNγ-captured T cells with a CD19CAR lentivirus followed by stimulation with CD19 antigen resulted in 50 to 70% of the CAR⁺ T cells responding to pp65 stimulation, representing the subset of functional CMV/CAR T cells. The CMV/CAR T cells exhibited specific cytolytic activity and secreted IFNγ, as well as proliferating vigorously after engagement of endogenous CMVpp65 T cell receptors or engineered CD19 CARs. Upon transfer into tumor bearing mice, the CMV/CAR T cells mediated cytokine released syndrome (CRS), which has been found to correlate with anti-tumor efficacy in the clinic.

While the CMV/CAR T cells described herein express a CAR targeted to CD19, the same methods can be used to generate CMV CAR T cells targeted to any desired antigen.

Efficient in vivo activation of virus-specific T cells through the TCR demands that viral antigens are processed and presented in a human leukocyte antigen (HLA)-dependent manner. This can be achieved by administering CMV Triplex Vaccine to the patient subsequent to administration of the CMV/CAR T cells.

The antitumor activity of CMV/CAR T cells can be enhanced as a consequence of proliferation following CMV peptide vaccination. This suggests that the cell dose of CMV/CAR T cells could be significantly decreased as compared to conventional CAR T cells, due to their potential to proliferate in vivo in response to vaccine, avoiding prolonged culture times and the risk of terminal differentiation.

In some cases, such as in the CMV/CAR T cells targeted to CD19 described herein, the CMV/CAR T cells also express a truncated EGFR (EGFRt). Cells expressing EGFRt can be killed by administration of an antibody, such a cetuximab, targeted to EGFR. This permits control and reduction of potential on/off-target toxicity.

The administration of CMV Triplex Vaccine subsequent to treatment with CMV/CD19 CAR T cells can augment the antitumor activity of adoptively transferred CMV/CD19 CAR T cells in several scenarios: 1) to salvage patients not achieving complete remission or relapsing after CAR T cell therapy, 2) vaccine boost when CD19 CAR T cells are failing to persist regardless of tumor responses at that time, 3) planned vaccination on days post-administration CD19 CAR T cells. There is also potential benefit of using the CMV/CAR T cells pre-emptively post-allogeneic HCT, both to eliminate minimal residual disease (MRD) and control CMV, potentially preventing reactivation of virus or undergoing expansion in response to latent CMV re-activation.

Moreover, administration of CMV Triplex Vaccine has the potential to profoundly impact the general field of adoptive T cell therapy, since by transducing a variety of tumor-directed CARs into CMV-specific T cells, it is possible to tailor this strategy to a wide range of malignancies and tumor targets.

### Triplex Vaccine

CMV Triplex Vaccine is a recombinant MVA that expresses three CMV antigens, i.e., at least a portion or Immediate-Early Gene-1 (IE1), at least a portion of Immediate-Early Gene-2 (IE2) and at least a portion of pp65. The IE1 antigen and the IE2 antigen can be expressed a fusion protein, for example, a protein encoded by the nucleotide sequence of SEQ ID NO: C. Expression of the CMV antigens can be under the control of a modified H5 (mH5) promoter. A CMV Triplex Vaccine is fully described in U.S. Pat. No. 8,580,276 and in Wang et al. (Vaccine 28:1547, 2010)

The CMV Triplex Vaccine can express CMV pp65 and an CMV IE fusion protein (IEfusion). The IEfusion can include an antigenic portion of IE1 (e.g., Exon 4) and an antigenic portion of 1E2 (e.g., Exon 5), wherein the antigenic portions elicit an immune response when expressed by a vaccine. In one aspect, the IEfusion is has the sequence encoded by SEQ ID NO: C or another nucleotide sequence that encodes the same amino acid sequence as SEQ ID NO: C.

As explained in U.S. Pat. No. 8,580,276, the CMV Triplex Vaccine includes three of the best recognized antigens in the CD8 subset: pp65, IE1, and IE2. There is no region of homology greater than 5 amino acids between the major exons of both proteins. Individually, both antigens are recognized broadly by almost 70% of the general population (Sylwester et al. 2005). The divergent sequence of both IE1/e4 and IE2/e5 used here predicts an entirely different subset of HLA binding peptides using publicly available Class I and II motif algorithms (Peters and Sette 2007). Human subjects that were evaluated for recognition of both IE1 and IE2 antigens were found in many instances to recognize one or the other but not both. Among the research subjects analyzed, 24% recognized IE2 with or without pp65 to the exclusion of IE1. This result strongly suggests that the recognition elements for both antigens are unique, and by including both of them in the vaccine, the breadth of individuals with disparate HLA types that will recognize and develop an immune response to the vaccine is extended. The fusion of major exons from both antigens achieves the dual goal of reducing the number of separate inserts and eliminating the need for a third insert promoter. The advantages of this approach include placement of all vaccine antigens in one vector, and diminishing the dose of virus needed to attain sufficient immunity simultaneously against all of the included antigens.

Also as explained in U.S. Pat. No. 8,580,276, prior to conducting experiments with rMVA in clinical samples, the capacity for stimulation of both CD4+ and CD8+ T cells was assessed using the commercially available pp65 and IE1 library and a newly designed IE2 peptide library. Relationships among the T cell populations were similar to prior results: pp65 promotes a substantial CD4 and CD8 response in over 70% of participants, while IE1 and IE2 are recognized less frequently and mainly in the CD8+ T cell compartment. MVA expressing the IEfusion antigen with or without the pp65 antigen was evaluated in PBMC from healthy volunteers to establish their recognition properties using a fully human system. The results showed that the memory T cell expansion stimulated by the rMVA for both the IEfusion and pp65 antigens, followed the proportions found ex vivo for the same volunteers using the peptide library approach. While there was substantial amplification of the relevant T cell populations, the stimulation did not skew the population towards a particular subset or antigen specificity. The data also confirms that the IEfusion protein is processed and presented appropriately to stimulate existing T cell populations in a manner that maintains the phenotypic distribution as expected in the ex vivo analysis. The most rigorous evaluation of the processing of the rMVA for T cell response is using PBMC from transplant patients. PBMC from HCT recipients in all three risk categories were evaluated and an equivalently strong recognition of both rMVAs was found. In some cases, it was even more vigorous than in the PBMC of healthy adults. No interference with the recognition of the IE antigen by the co-expressed pp65 antigen was found from the same rMVA, which further confirms that the recognition of both antigens can take place at the same time and derived from the same vector.

In one embodiment, the nucleic acid sequence encoding vaccinia mH5 promoter has a sequence containing nucleotides 3075-3168 of SEQ ID NO: A or 3022-3133 of SEQ ID NO: B

### Components of Chimeric Antigen Receptors

A wide variety of CAR have been described in the scientific literature. In general CAR include an extracellular antigen-binding domain (often a scFv derived from variable heavy and light chains of an antibody), a spacer domain, a transmembrane domain and an intracellular signaling domain. The intracellular signaling domain usually includes the endodomain of a T cell co-stimulatory molecule (e.g., CD28, 4-1BB or OX-40) and the intracellular domain of CD3ζ.

### Spacer Region

The CAR described herein can include a spacer region located between the cancer antigen targeting domain (e.g., a CD19 ScFv, e.g., the scFv portion can include the CD19 targeted scFv sequence of a CD19-targeted CAR such as that described in Wang et al. 2016 Blood 127:2980-2990) and the transmembrane domain. A variety of different spacer regions can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacer Regions

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + | 22 aa | ESKYGPPCPPCPGGGSSGGGSG (SEQ |
| linker | | ID NO: 5) |
| CD28 hinge | 39 aa | IEVMYPPPYLDNEKSNGTIIHVKGKHL |
| | | CPSPLFPGPSKP (SEQ ID NO: 6) |
| CD8 hinge-48 aa | 48 aa | AKPTTTPAPRPPTPAPTIASQPLSLRPE |
| | | ACRPAAGGAVHTRGLDFACD (SEQ |
| | | ID NO: 7) |
| CD8 hinge-45 aa | 45 aa | TTTPAPRPPTPAPTIASQPLSLRPEACR |
| | | PAAGGAVHTRGLDFACD (SEQ ID |
| | | NO: 8) |
| IgG4(HL-CH3) | 129 aa | ESKYGPPCPPCPGGGSSGGGSGGQPR |
| (includes S228P in hinge) | | EPQVYTLPPSQEEMTKNQVSLTCLVK |
| | | GFYPSDIAVEWESNGQPENNYKTTPP |
| | | VLDSDGSFFLYSRLTVDKSRWQEGNV |
| | | FSCSVMHEALHNHYTQKSLSLSLGK |
| | | (SEQ ID NO: 9) |
| IgG4 | 229 aa | ESKYGPPCPSCPAPEFEGGPSVFLFPPK |
| (L235E, N297Q) | | PKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK (SEQ ID |
| | | NO: 10) |
| IgG4 | 229 aa | ESKYGPPCPPCPAPEFEGGPSVFLFPPK |
| (S228P, L235E, N297Q) | | PKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK (SEQ ID |
| | | NO: 11) |
| IgG4 | 107 aa | GQPREPQVYTLPPSQEEMTKNQVSLT |
| (CH3) | | CLVKGFYPSDIAVEWESNGQPENNYK |
| | | TTPPVLDSDGSFFLYSRLTVDKSRWQ |
| | | EGNVFSCSVMHEALHNHYTQKSLSLS |
| | | LGK (SEQ ID NO: 12) |

Some spacer regions include all or part of an immunoglobulin (e.g., IgG1, IgG2, IgG3, IgG4) hinge region, i.e., the sequence that falls between the CH1 and CT2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one ore more amino acid modifications, for example, 1, 2, 3, 4 or 5 substitutions, e.g., substitutions that reduce off-target binding.

The spacer region can also comprise a IgG4 hinge region having the sequence ESKYGPPCPSCP (SEQ ID NO:4) or ESKYGPPCPPCP (SEQ ID NO:3).

The spacer region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGK (SEQ ID NO:12). Thus, the entire spacer region can comprise the sequence: ESKYGPPCPPCPGGGSSGGGSGGQPREPQVYTLPPSQEEMTKNQVSLTCLVKGFYPSDIA VEWESNGQPENNYKTTPPVLDSDGSFFLYSRLTVDKSRWQEGNVFSCSVMIIEALHNHY TQKSLSLSLGK (SEQ ID NO:11). In some cases, the spacer has 1, 2, 3, 4 or 5 single amino acid changes (e.g., conservative changes) compared to those shown in Table 1. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID |
| | | | NO: 13) |
| CD28 | NM_006139 | 27 aa | FWVLVVVGGVLACYSLLVTVAFIIFWV |
| | | | (SEQ ID NO: 14) |
| CD28(M) | NM_006139 | 28 aa | MFWVLVVVGGVLACYSLLVTVAFIIFWV |
| | | | (SEQ ID NO: 15) |
| CD4 | M35160 | 22 aa | MALIVLGGVAGLLLFIGLGIFF (SEQ ID |
| | | | NO: 16) |
| CD8tm | NM_001768 | 21 aa | IYIWAPLAGTCGVLLLSLVIT (SEQ ID |
| | | | NO: 17) |
| CD8tm2 | NM_001768 | 23 aa | IYIWAPLAGTCGVLLLSLVITLY (SEQ ID |
| | | | NO: 18) |
| CD8tm3 | NM_001768 | 24 aa | IYIWAPLAGTCGVLLLSLVITLYC (SEQ |
| | | | ID NO: 19) |
| 41BB | NM_001561 | 27 aa | IISFFLALTSTALLFLLFF LTLRFSVV (SEQ |
| | | | ID NO: 20) |

### Costimulatory Domain

The costimulatory domain can be any domain that is suitable for use with a CD3 signaling domain. In some cases, the costimulatory domain is a CD28 costimulatory domain that includes a sequence that is at least 90%, at least 95%, at least 98% identical to or identical to: RSKRSRGGHSDYMINIMTPRRPGPTRKHYQPYAPPRDFAAYRS (SEQ ID NO:23; LL to GG amino acid change double underlined). In some cases, the CD28 co-signaling domain has 1, 2, 3, 4 of 5 amino acid changes (preferably conservative and preferably not in the underlined GG sequence) compared to SEQ ID NO:23. In some cases the co-signaling domain is a 4-11B1 co-signaling domain that includes a sequence that is at least 9000, at least 9500, at least 9800 identical to or identical to: KRGRKKLLYIFKQPFNRWVQTTQEEDGCSCRFPEEEEGGCEL (SEQ ID NO:24). In some cases, the 4-11B1 co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD32 Domain and Examples of Costimulatory Domains

| Name | Accession | Length | Sequence |
|---|---|---|---|
| CD3 | J04132.1 | 113 aa | RVKFSRSADAPAYQQGQNQLYNELNLGR |
| | | | REEYDVLDKRRGRDPEMGGKPRRKNPQ |
| | | | EGLYNELQKDKMAEAYSEIGMKGERRR |
| | | | GKGHDGLYQGLSTATKDTYDALHMQAL |
| | | | PPR (SEQ ID NO: 21) |
| CD28 | NM_006139 | 42 aa | RSKRSRLLHSDYMNMTPRRPGPTRKHYQ |
| | | | PYAPPRDFAAYRS (SEQ ID NO: 22) |
| CD28gg* | NM_006139 | 42 aa | RSKRSRGGHSDYMNMTPRRPGPTRKHY |
| | | | QPYAPPRDFAAYRS (SEQ ID NO: 23) |
| 41BB | NM_001561 | 42 aa | KRGRKKLLYIFKQPFMRPVQTTQEEDGC |
| | | | SCRFPEEEEGGCEL (SEQ ID NO: 24) |
| OX40 | | 42 aa | ALYLLRRDQRLPPDAHKPPGGGSFRTPIQ |
| | | | EEQADAHSTLAKI (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO:21). In some cases, the CD3ζ signaling has 1, 2, 3, 4 of 5 amino acid changes (preferably conservative) compared to SEQ ID NO:21.

### Truncated EGFR

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVT SLLLCELPHPAFLLIPRKVCNGIGIGEFKDSLSINATNIKHFKNCTSISGDLHILPVAFR GDSFTHTPPLDPQELDILKTVKEITGFLLIQAWPENRTDLHAFENLEIIRGRTKQHGQFSL AVVSLNITSLGLRSLKEISDGDVIISGNKNLCYANTINWKKLFGTSGQKTKIISNRGENSC KATGQVCHALCSPEGCWGPEPRDCVSCRNVSRGRECVDKCNLLEGEPREFVENSECIQC HPECLPQAMNITCTGRGPDNCIQCAHYIDGPHCVKTCPAGVMGENNTLVWKYADAGH VCHLCHPNCTYGCTGPGLEGCPTNGPKIPSIATGMVGALLLLLVVALGIGLFM (SEQ ID NO:28). In some cases, the truncated EGFR has 1, 2, 3, 4 of 5 amino acid changes (preferably conservative) compared to SEQ ID NO:28.

### Example 1: Enrichment of CMV-Specific T Cells from PBMC of Healthy Donors after Stimulation with cGMP Grade CMVpp65 Protein

CMV-specific T cells were prepared from PBMC of healthy donors by stimulating the PBMC with cGMP grade CMVpp65 protein. Briefly, PBMCs were isolated by density gradient centrifugation over Ficoll-Paque (Pharmacia Biotech, Piscataway, NJ) from peripheral blood of consented healthy, HLA-A2 CMV-immune donors under a City of Hope Internal Review Board-approved protocol. PBMC were frozen for later use. After overnight rest in RPMI medium containing 5% Human AB serum (Gemini Bio Products) without cytokine, the PBMC were stimulated with current good manufacturing practice (cGMP) grade CMVpp65 protein (Miltenyi Biotec, Germany) at 10 ul/10×10⁶ cells for 16 hours in RPMI 1640 (Irvine Scientific, Santa Ana, CA) supplemented with 2 mM L-glutamine (Irvine Scientific), 25 mM N-2-hydroxyethylpiperazine-N-2-ethanesulfonic acid (HEPES, Irvine Scientific), 100 U/mL penicillin, 0.1 mg/mL streptomycin (Irvine Scientific) in the presence of 5U/ml IL-2 and 10% human AB serum. CMV-specific T cells were selected using the IFNγ capture (Miltenyi Biotec, Germany) technique according to the manufacturer's instructions.

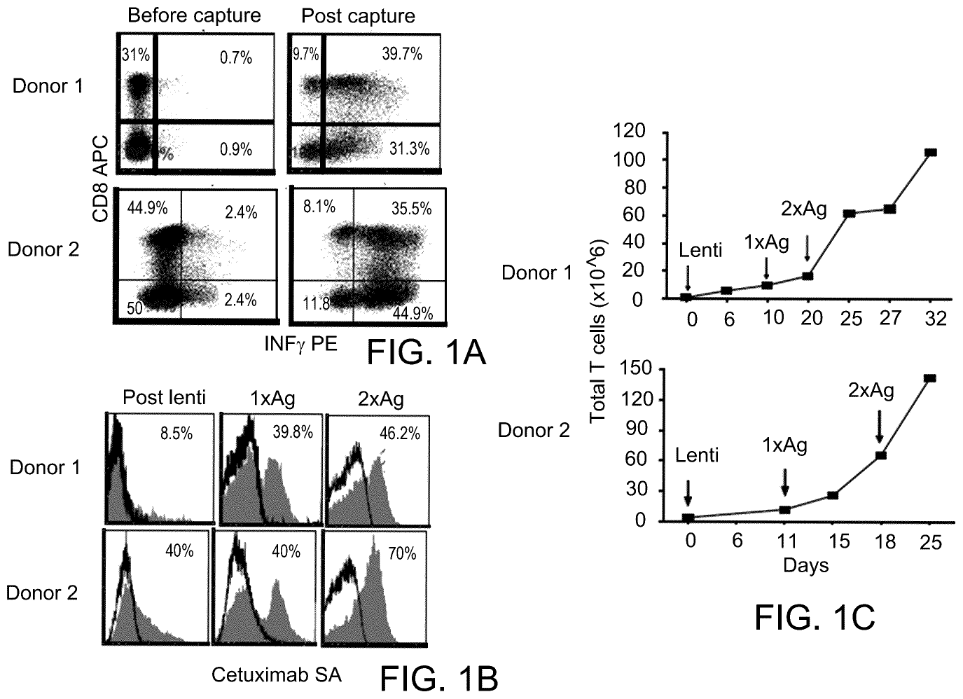

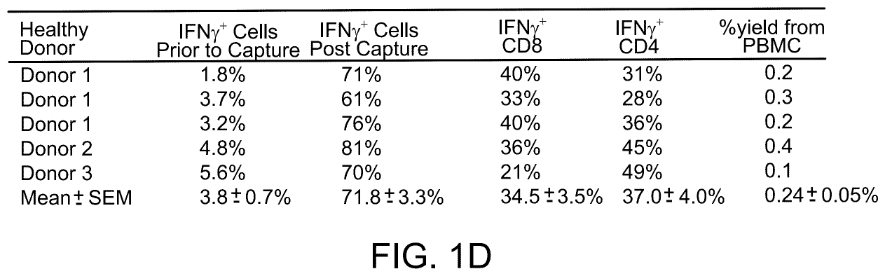

To demonstrate the consistency of this clinically feasible process, the selection was repeated five times using PBMC from three different donors. IFNγ-positive T cells were consistently enriched from a baseline mean of 3.8% (range 1.8-5.6) to a post-capture mean of 71.8% (range 61-81) and contained polyclonal CD8⁺ (34%) and CD4⁺ T cells (37%) after selection (FIG. 1A and FIG. 1C). Moreover, the selected CMV-specific T cells included both CD4 and CD8 subsets and represented the entire spectrum of CMV-specificity, showing responsiveness to CMVpp65 pepmix stimulation with broad recognition.

### Example 2: Genetic Modification of Enriched CMV-Specific T Cells to Express CD19 CAR and In Vitro Expansion of the CMV/CAR T Cells

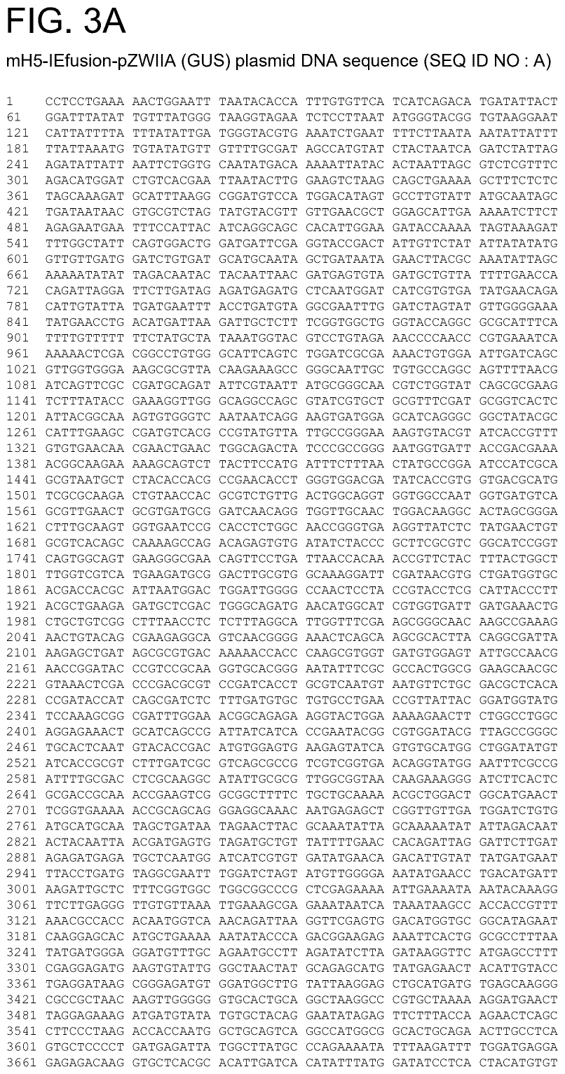

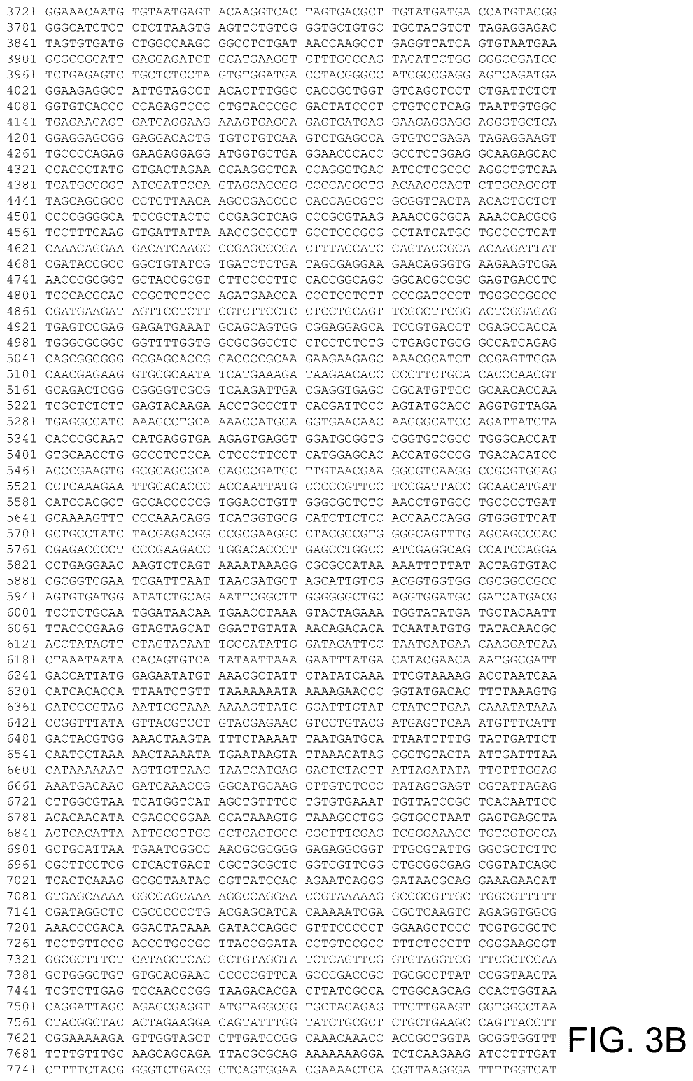

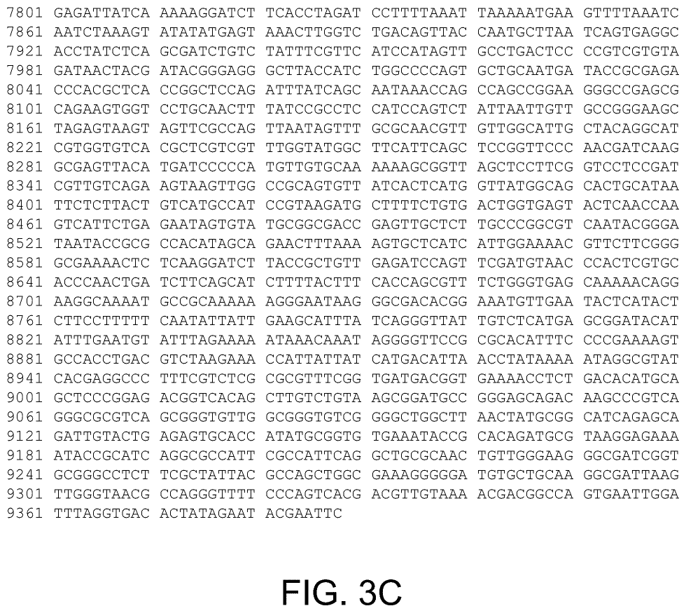

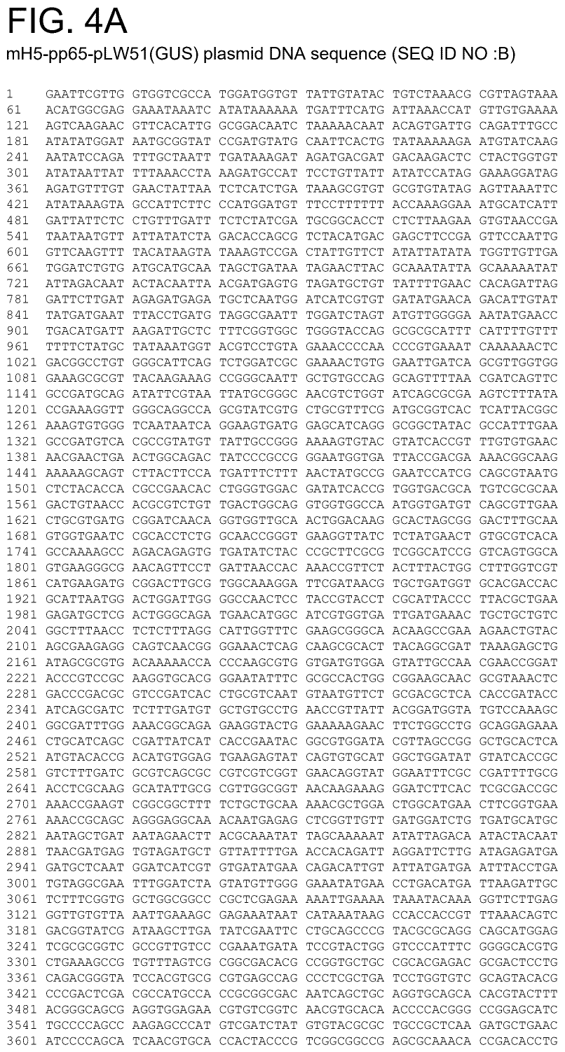

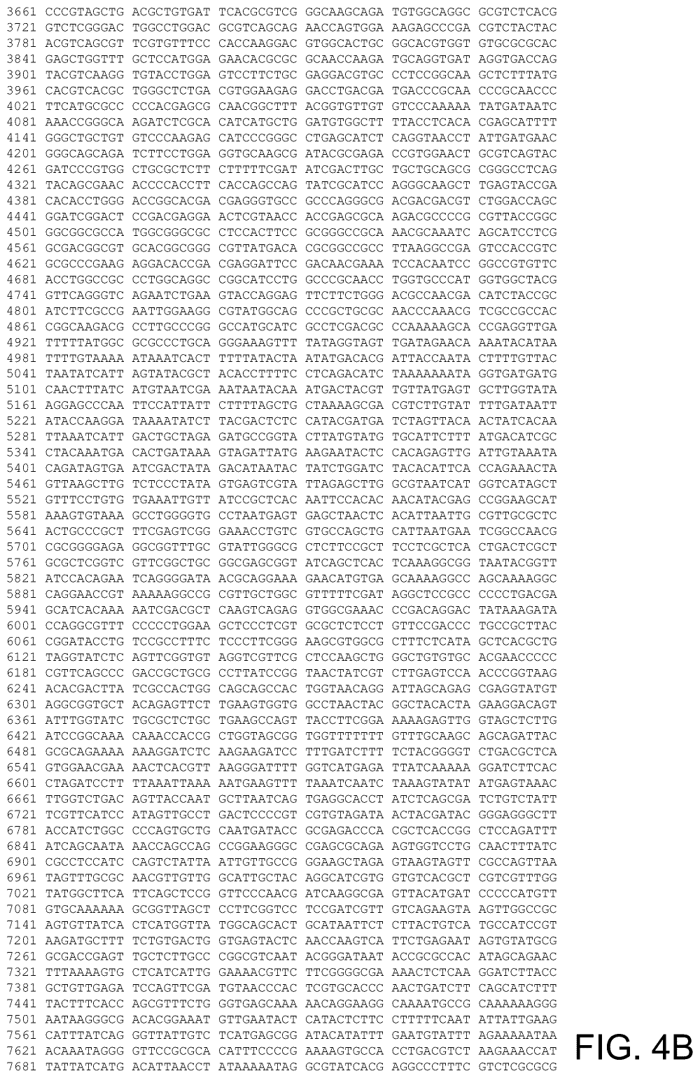

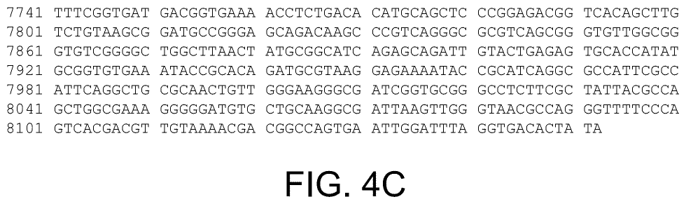

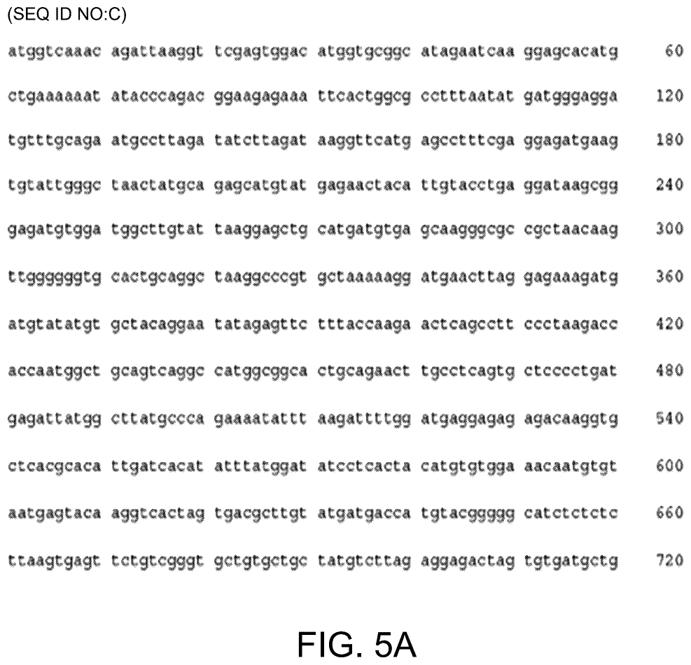

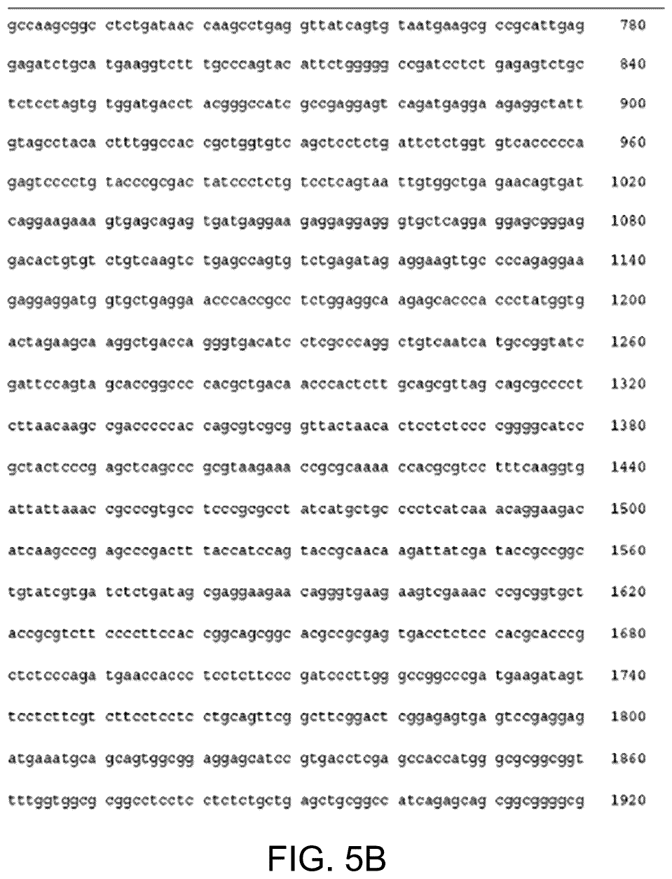

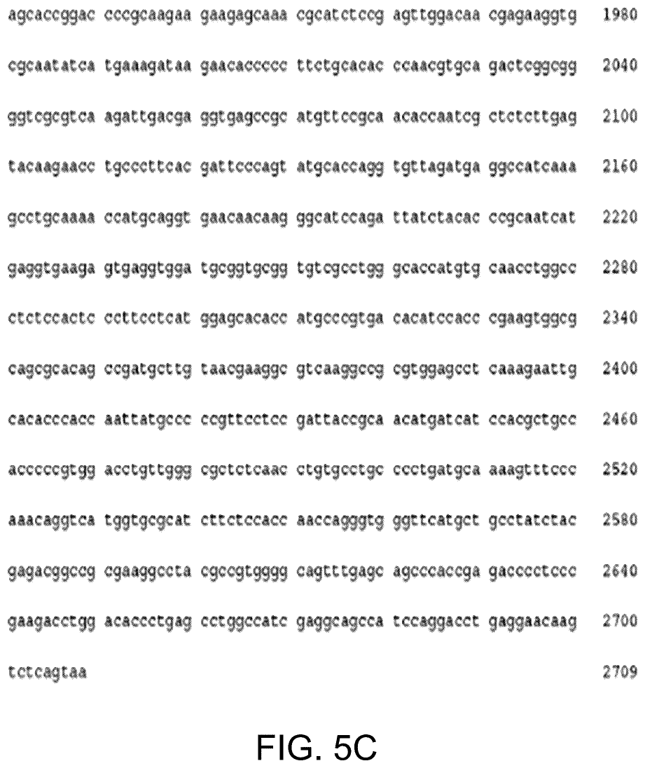

In the clinically adaptable procedure, IFNγ-captured CMV-specific T cells were transduced 2 days after the selection, without OKT3 activation, using the second generation CD19RCD28EGFRt lentiviral construct containing the IgG4 Fc hinge region mutations (L235E; N297Q) that improve potency due to distortion of the FcR binding domain. The complete amino sequence of the this CD19 CAR is depicted in FIGS. 3A-3C. Starting seven days post lenti-transduction, the cells were stimulated on a weekly basis with 8000 cGy-irradiated, CD19-expressing NIH3T3 cells at a 1:10 ratio (T cells: CD19NIH 3T3). The percentage of CAR⁺ cells detected by cetuximab increased from 8% post transduction to 46% after 2 rounds of stimulation with a 120-150-fold total cell increase (FIG. 1B and FIG. 1D). Further details regarding the lentiviral construct, the CD19-expressing NIH3T3 cells and other materials and techniques used in the studies described herein are presented below.

### Example 3: CMV/CAR T Cells Exhibited Specific Effector Function after Stimulation Through Pre-Defined Viral TCR and CD19CAR

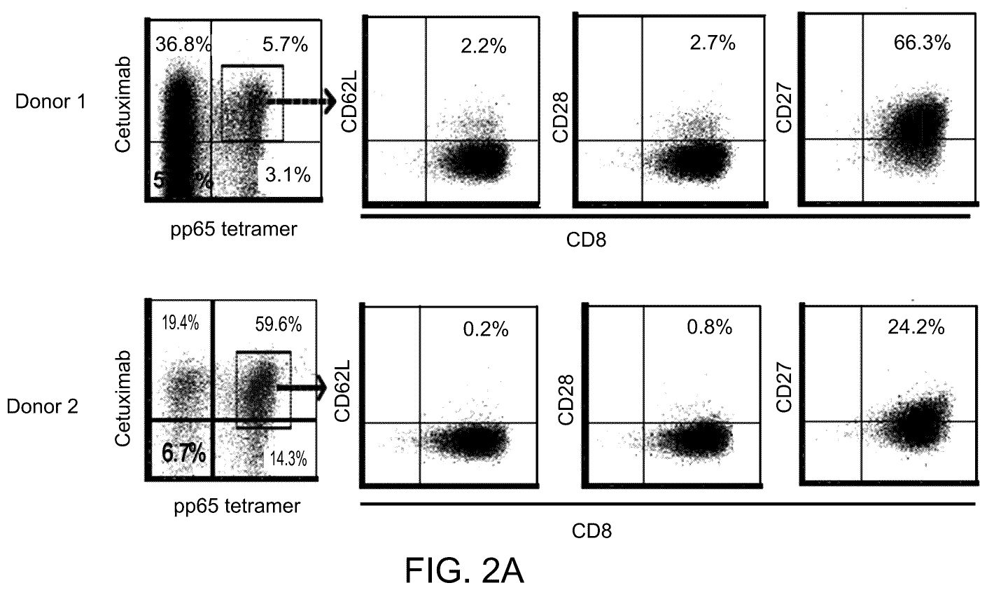

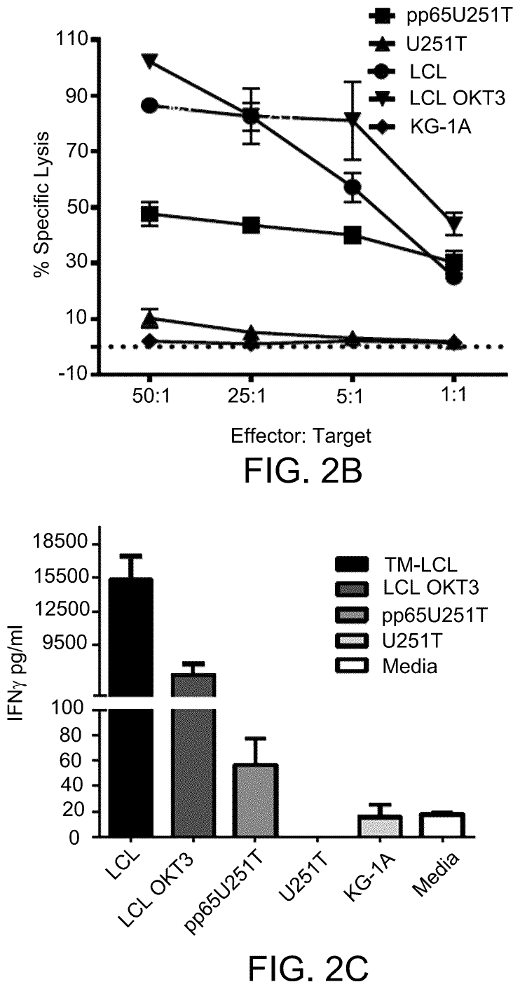

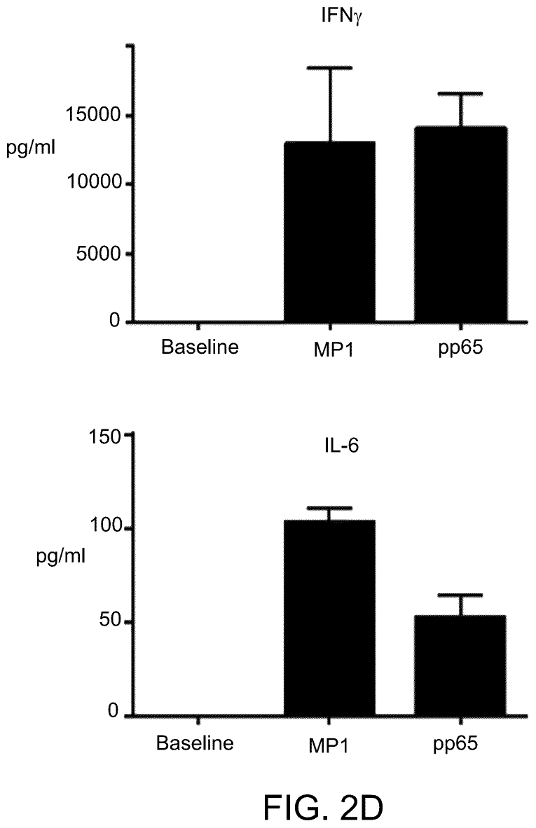

Recapitulating our previous studies (23), the ex vivo expanded CMV-specific T cells possessed an effector phenotype and no longer expressed the central memory markers of the originally selected cells, such as CD62L, CD28, and IL-7Ra (FIG. 2A and FIG. 2D). However, levels of CD27 remained high, suggesting a greater proliferative potential that has been associated with greater clinical efficacy (24). To investigate CMV/CAR T cell effector function via signaling by both the endogenous CMV-specific TCR and the introduced CD19CAR, we evaluated response to engineered pp65-expressing U251T cells from HLA-A2 donors, and also allogeneic CD19⁺LCLs, based on cytotoxicity, cytokine production and proliferation profiles. As expected, the expanded CMV/CAR T cells specifically lysed CD19⁺LCLs with the same maximum killing levels as the OKT3-expressing LCL used as positive controls. Likewise, specific killing was also observed when pp65U251T cells were used as targets as compared to parental U251T cells (FIG. 2B). Accordingly, after overnight stimulation, elevated IFNγ secretion was observed after either CD19 or pp65 antigen stimulation as compared to antigen-negative stimulators such as KG1a and U251T parental cells (FIG. 2C).

Antibodies and Flow Cytometry: Fluorochrome-conjugated isotype controls, anti-CD3, anti-CD4, anti-CD8, anti-CD28, anti-CD45, anti-CD27, anti-CD62L, anti-CD127, anti-IFN□, and streptavidin were obtained from BD Biosciences. Biotinylated cetuximab was generated from cetuximab purchased from the City of Hope pharmacy. The IFN-□ Secretion Assay—Cell Enrichment and Detection Kit and CMVpp65 protein were purchased from Miltenyi Biotec (Miltenyi Biotec, Germany). Phycoerythrin (PE)-conjugated CMV pp65 (NLVPMVATV)-HLA-A2*0201 iTAg MHC tetramer, PE-conjugated multi-allele negative tetramer was obtained from Beckman Coulter (Fullerton, CA). Carboxyfluorescein diacetate succinimidyl ester (CFSE) was purchased from Invitrogen (Carlsbad, CA). All monoclonal antibodies, tetramers and CFSE were used according to the manufacturer's instructions. Flow cytometry data acquisition was performed on a MACSQuant (Miltenyi Biotec, Germany) or FACScalibur (BD Biosciences), and the percentage of cells in a region of analysis was calculated using FCS Express V3 (De Novo Software).

Cell lines: EBV-transformed lymphoblastoid cell lines (LCLs) were made from peripheral blood mononuclear cells (PBMC) as previously described (16). To generate LCL-OKT3, allogeneic LCLs were resuspended in nucleofection solution using the Amaxa Nucleofector kit T, OKT3-2A-Hygromycin_pEK plasmid was added to 5 μg/107 cells, the cells were electroporated using the Amaxa Nucleofector I, and the resulting cells were grown in RPMI 1640 with 10% FCS containing 0.4 mg/ml hygromycin. To generate firefly luciferase+ GFP+ LCLs (fflucGFPLCLs), LCLs were transduced with lentiviral vector encoding eGFP-ffluc. Initial transduction efficiency was 48.5%, so the GFP+ cells were sorted by FACS for >98% purity. To generate CD19 NIH3T3 cells, parental NIH3T3 cells (ATCC) were transduced with a retrovirus encoding CD80, CD54 and CD58 (17). The established cell line was further engineered to express CD19GFP by lentiviral transduction. GFP+ cells were purified by FACS sorting and expanded for the use of stimulation of CMV/CAR T cells. To generate pp65 stimulator cells, U251T cells derived from human glioblastoma cells from an HLA A2 donor (ATCC) were transduced with a lentiviral vector encoding full length pp65 fused to green fluorescent protein (GFP). pp65U251T cells were purified by GFP expression using flow cytometry. Banks of all cell lines were authenticated for the desired antigen/marker expression by flow cytometry prior to cryopreservation, and thawed cells were cultured for less than 6 months prior to use in assays.

Peptides: The pp65 peptide NLVPMVATV (HLA-A 0201 CMVpp65) at >90% purity was synthesized using automated solid phase peptide synthesis in (Department of Experimental Therapeutics, Beckman Research Institute of City of Hope). MP1 GIGFVFTL peptide (HLA-A 0201 influenza) was synthesized at the City of Hope DNA/RNA Peptide Synthesis Facility, (Duarte, CA). pepMix HCMVA (pp65) (pp65pepmix) was purchased from JPT peptide Technologies (GmbH, Berlin Germany). All peptides were used according to the manufacturer's instructions.

Lentivirus vector construction: The lentivirus CAR construct was modified from the previously described CD19-specific scFvFc:ζ chimeric immunoreceptor(18), to create a third-generation vector. The CD19CAR containing a CD28□ co-stimulatory domain carries mutations at two sites (L235E; N297Q) within the CH2 region on the IgG4-Fc spacers to ensure enhanced potency and persistence after adoptive transfer (FIG. 7). The lentiviral vector also expressed a truncated human epidermal growth factor receptor (huEGFRt), which includes a cetuximab (Erbitux™) binding domain but excludes the EGF-ligand binding and cytoplasmic signaling domains. A T2A ribosome skip sequence links the codon-optimized CD19R:CD28:ζ sequence to the huEGFRt sequence, resulting in coordinate expression of both CD19R:CD28:ζ and EGFRt from a single transcript (CD19CARCD28EGFRt) (19). The CD19RCD28EGFRt DNA sequence (optimized by GeneArt) was then cloned into a self-inactivating (SIN) lentiviral vector pHIV7 (gift from Jiing-Kuan Yee, Beckman Research Institute of City of Hope) in which the CMV promoter was replaced by the EF-1α promoter.

Enrichment of CMV-specific T cells after CMVpp65 protein stimulation: PBMCs were isolated by density gradient centrifugation over Ficoll-Paque (Pharmacia Biotech, Piscataway, NJ) from peripheral blood of consented healthy, HLA-A2 CMV-immune donors under a City of Hope Internal Review Board-approved protocol. PBMC were frozen for later use. After overnight rest in RPMI medium containing 5% Human AB serum (Gemini Bio Products) without cytokine, the PBMC were stimulated with current good manufacturing practice (cGMP) grade CMVpp65 protein (Miltenyi Biotec, Germany) at 10 μl/10×10⁶ cells for 16 hours in RPMI 1640 (Irvine Scientific, Santa Ana, CA) supplemented with 2 mM L-glutamine (Irvine Scientific), 25 mM N-2-hydroxyethylpiperazine-N′-2-ethanesulfonic acid (HEPES, Irvine Scientific), 100 U/mL penicillin, 0.1 mg/mL streptomycin (Irvine Scientific) in the presence of 5U/ml IL-2 and 10% human AB serum. CMV-specific T cells were selected using the IFNγ capture (Miltenyi Biotec, Germany) technique according to the manufacturer's instructions.

Derivation and expansion of CMV/CAR T cells: The selected CMV-specific T cells were transduced on day 2 post IFNγ capture with lentiviral vector expressing CD19CARCD28EGFRt at MOI 3. Seven to ten days after lenti-transduction, the CMV/CAR T cells were expanded by stimulation through CAR-mediated activation signals using 8000 cGy-irradiated CD19-expressing NIH 3T3 cells at a 10:1 ratio (T cells:CD19 NIH3T3) once a week as described (17) in the presence of IL-2 50U/ml and IL-15 1 ng/ml. After 2 rounds of expansion, the growth and functionality of the CMV/CAR T cells was evaluated in vitro and in vivo.

Intracellular cytokine staining: CMV/CAR T cells (10⁵) were activated overnight with 105 LCL-OKT3, LCL, or KG1a cells in 96-well tissue culture plates, and with 10⁵ U251T and engineered pp65-expressing U251T cells (pp65U251T) in 24-well tissue culture plates in the presence of Brefeldin A (BD Biosciences). The cell mixture was then stained using anti-CD8, cetuximab and streptavidin, and pp65Tetramer to analyze surface co-expression of CD8, CAR and CMV-specific TCR, respectively. Cells were then fixed and permeabilized using the BD Cytofix/Cytoperm kit (BD Biosciences). After fixation, the T cells were stained with an anti-IFNγ.

CFSE Proliferation assays: CMV/CAR T cells were labeled with 0.5 μM CFSE and co-cultured with stimulator cells LCL-OKT3, LCLs, and pp65 U251T for 8 days. Co-cultures with U251T and KG1a cells were used as negative controls. Proliferation of CD3- and CAR-positive populations was determined using multicolor flow cytometry.

Cytokine production assays: T cells (105) were co-cultured overnight in 96-well tissue culture plates with 105 LCL-OKT3, LCL, or KG1a cells and in 24-well tissue culture plates with 105 U251T and engineered pp65-expressing U251T cells. Supernatants were then analyzed by cytometric bead array using the Bio-Plex Human Cytokine 17-Plex Panel (Bio-Rad Laboratories) according to the manufacturer's instructions.

Cytotoxicity assays: 4-hour chromium-release assays (CRA) were performed as previously described (20) using effector cells that had been harvested directly after 2 rounds of CD19 Ag stimulations.

What is claimed is:

1. A method for treating a patient comprising: (a) providing a composition comprising a population of T cells expressing both a chimeric antigen receptor (CAR) and a T cell receptor specific for a cytomegalovirus (CMV) antigen; (b) administering the composition to the patient; and (c) administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient.

2. The method of claim 1 wherein the step of administering a viral vector to the patient comprises administering recombinant MVA virus.

3. The method of claim 2 wherein expression of (i) CMV pp65 and (ii) the fusion protein comprising exon 4 of CMV protein IE1 (e4) and exon 5 of CMV protein IE2 (e5) is under the control of mH5 promoter.

4. The method of claim 1 wherein the patient is immunocompromised.

5. The method of claim 1 wherein the patient receives immunosuppressive therapy.

6. The method of claim 1 wherein the patient is immunocompetent.

7. The method of claim 1 wherein the patient is CMV-seronegative prior to treatment.

8. The method of claim 1 wherein the patient is CMV-seropositive prior to treatment.

9. The method of claim 1 wherein the patient received hematopoietic stem cells (HSC) from a CMV-positive or CMV-negative donor prior to administering the comprising a population of T cells expressing both a chimeric antigen receptor (CAR) and a T cell receptor specific for a cytomegalovirus (CMV) antigen.

10. The method of claim 9 wherein the CAR is targeted to CD19.

11. The method of claim 9 wherein the hematopoietic stem cells were autologous to the patient.

12. The method of claim 9 wherein the hematopoietic stem cells were allogenic to the patient.

13. The method of any of claims 9-12 wherein the patient is suffering from non-Hodgkin's Lymphoma.

14. The method of claim 1 wherein administration of the viral vector occurs at least 5 days after treatment with the composition comprising a population of T cells.

15. The method of claim 1 wherein the viral vector is administered to the patient both prior to and subsequent to the administration of the composition comprising a population of T cells.

16. The method of claim 9 wherein the viral vector is administered to the hematopoietic stem cell donor prior to harvesting the stem cells.

17. The method of claim 1 wherein the viral vector is administered to the patient only prior to the administration of the composition comprising a population of T cells.

18. The method of claim 1 wherein the viral vector is administered to the patient only subsequent to the administration of the composition comprising a population of T cells.

19. The method of claim 9 wherein the viral vector is administered to the patient or the hematopoietic stem cell transplant donor at least twice subsequent to the administration of the composition comprising a population of T cells.

20. The method of claim 1 wherein the viral vector is administered to the patient at least four times.

21. The method of claim 1, wherein the step of providing a population of T cells expressing a CAR and a T cell receptor specific for a CMV antigen comprises: (a) providing PBMC or a T cell subpopulation from a CMV-seropositive human donor; (b) exposing the PBMC or the T cell subpopulation to at least one CMV antigen; (c) treating the exposed cells to produce a population of cells enriched for stimulated cells specific for CMV; (d) transducing at least a portion of the enriched population of cells with a vector expressing a CAR.

22. The method of claim 21, wherein the step of treating the exposed cells to produce a population of cells enriched for stimulated cells specific for CMV comprises treating the stimulated cells to produce a population of cells enriched for cells expressing an activation marker.

23. The method of claim 1, wherein the step of providing a population of T cell expressing a CAR and a T cell receptor specific for a CMV antigen comprises: (a) administering a viral vector encoding: (i) CMV pp65 and (ii) a fusion protein comprising exon 4 of CMV protein IE1 (e4) and exon 5 of CMV protein IE2 (e5) to a human donor to convert a CMV-seronegative human donor to one containing T cells responsive to CMV antigens pp65, IE1 and IE2; (b) obtaining PBMC from the CMV-seropositive human donor; (c) exposing the PBMC to at least one CMV antigen; (d) treating the exposed cells to produce a population of cells enriched for stimulated cells specific for CMV; (e) transducing at least a portion of the enriched population of cells with a vector expressing a CAR, thereby providing a population of T cell expressing a CAR and a T cell receptor specific for a CMV antigen.

24. The method of claim 1, wherein the step of providing a population of T cell expressing a CAR and a T cell receptor specific for a CMV antigen comprises: (a) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

25. A method for preparing T cells expressing a CAR and a T cell receptor specific for a CMV antigen, the method comprising: (a)) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

26. A method for preparing T cells expressing a CAR and a T cell receptor specific for a CMV antigen, the method comprising: a)) administering a viral vector encoding: (i) CMV pp65 and (ii) a fusion protein comprising exon 4 of CMV protein IE1 (e4) and exon 5 of CMV protein IE2 (e5) to a CMV-positive human donor; (b) obtaining PBMC from the CMV-seropositive human donor; (b) exposing the PBMC to at least one CMV antigen; (c) treating the exposed cells to produce a population of cells enriched for stimulated cells specific for CMV; (d) transducing at least a portion of the enriched population of cells with a vector expressing a CAR, thereby providing a population of T cell expressing a CAR and a T cell receptor specific for a CMV antigen.

27. The method of any of claims 21-26, further comprising expanding the population of T cell expressing a CAR and a T cell receptor specific for a CMV antigen.

28. The method of claim 22, wherein the activation marker is IFN-γ or other activation marker such as CD137, CD107 or other cytokines.

29. The method of any of claims 21-26, wherein the CMV antigen is pp65 protein or an antigenic portion thereof.

30. The method of any of claims 21-26, wherein the CMV antigen comprises two or more different antigenic CMV pp65 peptides.

31. The method of any of claims 21-26, wherein the step of transducing the enriched population of cells does not comprise CD3 stimulation.

32. The method of any of claims 21-26, wherein the step of transducing the enriched population of cells does not comprise CD28 stimulation.

33. The method of any of claims 21-26, wherein the step of transducing the enriched population of cells does not comprise CD28 stimulation or CD3 stimulation.

34. The method of any of claims 21-26, wherein the step of transducing the enriched population of cells does not comprise exposing the cells to an anti-CD28 antibody or an anti-CD3 antibody.

35. The method of any of claims 21-26, wherein the enriched population of cells is at least 40% IFN-γ positive, at least 20% CD8 positive, and at least 20% CD4 positive.

36. The method of any of claims 21-26, wherein the enriched population of cells are cultured for fewer than 10 days prior to the step of transducing the enriched population of cells with a vector encoding a CAR.

37. The method of any of claims 21-26, further comprising expanding the CMV specific T cells expressing a CAR cells by exposing them to an antigen that binds to the CAR.

38. The method of any of claims 21-26, wherein the step of expanding the CMV-specific T cells expressing a CAR comprises exposing the cells to T cells expressing the antigen that binds the CAR.

39. The method of any of claims 21-26, wherein the expansion takes place is the presence of at least one exogenously added interleukin.

40. The method of claim 1 wherein the CAR is selective for an antigen selected from: CD19, CS1, CD123, 5T4, 8H9, αvβ6 integrin, alphafetoprotein (AFP), B7-H6, CA-125 carbonic anhydrase 9 (CA9), CD19, CD20, CD22, CD30, CD33, CD38, CD44, CD44v6, CD44v7/8, CD52, CD123, CD171, carcinoembryonic antigen (CEA), EGFrvIII, epithelial glycoprotein-2 (EGP-2), epithelial glycoprotein-40 (EGP-40), ErbB1/EGFR, ErbB2/HER2/neu/EGFR2, ErbB3, ErbB4, epithelial tumor antigen (ETA), FBP, fetal acetylcholine receptor (AchR), folate receptor-α, G250/CAIX, ganglioside 2 (GD2), ganglioside 3 (GD3), HLA-A1, HLA-A2, high molecular weight melanoma-associated antigen (HMW-MAA), IL-13 receptor α2, KDR, k-light chain, Lewis Y (LeY), L1 cell adhesion molecule, melanoma-associated antigen (MAGE-A1), mesothelin, Murine CMV infected cella, mucin-1 (MUC1), mucin-16 (MUC16), natural killer group 2 member D (NKG2D) ligands, nerve cell adhesion molecule (NCAM), NY-ESO-1, Oncofetal antigen (h5T4), prostate stem cell antigen (PSCA), prostate-specific membrane antigen (PSMA), receptor-tyrosine kinase-like orphan receptor 1 (ROR1), TAA targeted by mAb IgE, tumor-associated glycoprotein-72 (TAG-72), tyrosinase, and vascular endothelial growth factor (VEGF) receptors.

41. The method of claim 1, wherein the CAR is selective for an antigen selected from: CD19, CD123, CS1, BCMA, CD44v6, CD33, CD22, IL-13α2, PSA, HER-2, EGFRv3, CEA, and C7R.

42. The method of claim 1, wherein the CAR comprises: a scFv selective for the selected non-CMV antigen; a hinge/linker region; a transmembrane domain; a co-signaling domain; and CD3ζ signaling domain.

43. The method of claim 42, wherein the co-signaling domain is selected from a CD28 co-signaling domain and a 4-IBB co-signaling domain.

44. The method of claim 42, wherein the transmembrane domain is selected from a CD28 transmembrane domain and a CD4 transmembrane domain.

45. The method of claim 1 wherein the population of human T cells is autologous to the patient.

46. The method of claim 1 wherein the population of human T cells is allogeneic to the patient.

47. The method of claim 1 wherein the method reduces the risk of CMV infection.

48. The method of claim 1 wherein the method reduces CMV viremia and/or disease.

49. The method of claim 1 wherein the patient was CMV-immune prior to treatment and the method reduces the risk of CMV infection.

50. The method of claim 1 wherein the patient was not CMV-immune prior to treatment and the method reduces CMV viremia or disease.


SEQUENCE LISTING (31 sequences)

SEQ ID NO: 1 - skipped

SEQ ID NO: 2 - protein, 10 aa, Synthetic construct
GGGSSGGGSG

SEQ ID NO: 3 - protein, 12 aa, Synthetic construct
ESKYGPPCPPCP

SEQ ID NO: 4 - protein, 12 aa, Homo sapiens
ESKYGPPCPSCP

SEQ ID NO: 5 - protein, 22 aa, Synthetic construct
ESKYGPPCPPCPGGGSSGGGSG

SEQ ID NO: 6 - protein, 39 aa, Synthetic construct
IEVMYPPPYLDNEKSNGTIIHVKGKHLCPSPLFPGPSKP

SEQ ID NO: 7 - protein, 48 aa, Synthetic construct
AKPTTTPAPRPPTPAPTIASQPLSLRPEACRPAAGGAVHTRGLDFACD

SEQ ID NO: 8 - protein, 45 aa, Synthetic construct
TTTPAPRPPTPAPTIASQPLSLRPEACRPAAGGAVHTRGLDFACD

SEQ ID NO: 9 - protein, 129 aa, Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Synthetic construct
LCYLLDGILFIYGVILTALFL

SEQ ID NO: 14 - protein, 27 aa, Synthetic construct
FWVLVVVGGVLACYSLLVTVAFIIFWV

SEQ ID NO: 15 - protein, 28 aa, Synthetic construct
MFWVLVVVGGVLACYSLLVTVAFIIFWV

SEQ ID NO: 16 - protein, 22 aa, Synthetic construct
MALIVLGGVAGLLLFIGLGIFF

SEQ ID NO: 17 - protein, 21 aa, Synthetic construct
IYIWAPLAGTCGVLLLSLVIT

SEQ ID NO: 18 - protein, 23 aa, Synthetic construct
IYIWAPLAGTCGVLLLSLVITLY

SEQ ID NO: 19 - protein, 24 aa, Synthetic construct
IYIWAPLAGTCGVLLLSLVITLYC

SEQ ID NO: 20 - protein, 27 aa, Synthetic construct
IISFFLALTSTALLFLLFFLTLRFSVV

SEQ ID NO: 21 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Synthetic construct
RSKRSRLLHSDYMNMTPRRPGPTRKHYQPYAPPRDFAAYRS

SEQ ID NO: 23 - protein, 41 aa, Synthetic construct
RSKRSRGGHSDYMNMTPRRPGPTRKHYQPYAPPRDFAAYRS

SEQ ID NO: 24 - protein, 42 aa, Synthetic construct
KRGRKKLLYIFKQPFMRPVQTTQEEDGCSCRFPEEEEGGCEL

SEQ ID NO: 25 - protein, 42 aa, Synthetic construct
ALYLLRRDQRLPPDAHKPPGGGSFRTPIQEEQADAHSTLAKI

SEQ ID NO: 26 - skipped

SEQ ID NO: 27 - protein, 24 aa, Synthetic construct
LEGGGEGRGSLLTCGDVEENPGPR

SEQ ID NO: 28 - protein, 354 aa, Synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - DNA, 9388 bp, Synthetic construct
  source 1..9388: mol_type = other DNA
cctcctgaaaaactggaatttaatacaccatttgtgttcatcatcagacatgatattactggatttatattgtttatgggtaaggtagaatctccttaatatgggtacggtgtaaggaatcattattttatttatattgatgggtacgtgaaatctgaattttcttaataaatattatttttattaaatgtgtatatgttgttttgcgatagccatgtatctactaatcagatctattagagatattattaattctggtgcaatatgacaaaaattatacactaattagcgtctcgtttcagacatggatctgtcacgaattaatacttggaagtctaagcagctgaaaagctttctctctagcaaagatgcatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttggctattcagtggactggatgattcgaggtaccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacgccaccacaatggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgcacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaaaataaaggcgcgccataaaaatttttatactagtgtaccgcggtcgaatcgatttaattaacgatgctagcattgtcgacggtggtggcgcggccgccagtgtgatggatatctgcagaattcggcttggggggctgcaggtggatgcgatcatgacgtcctctgcaatggataacaatgaacctaaagtactagaaatggtatatgatgctacaattttacccgaaggtagtagcatggattgtataaacagacacatcaatatgtgtatacaacgcacctatagttctagtataattgccatattggatagattcctaatgatgaacaaggatgaactaaataatacacagtgtcatataattaaagaatttatgacatacgaacaaatggcgattgaccattatggagaatatgtaaacgctattctatatcaaattcgtaaaagacctaatcaacatcacaccattaatctgtttaaaaaaataaaaagaacccggtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattgactacgtggaaactaagtatttctaaaattaatgatgcattaatttttgtattgattctcaatcctaaaaactaaaatatgaataagtattaaacatagcggtgtactaattgatttaacataaaaaatagttgttaactaatcatgaggactctacttattagatatattctttggagaaatgacaacgatcaaaccgggcatgcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactatagaatacgaattc

SEQ ID NO: 30 - DNA, 8152 bp, Synthetic construct
  source 1..8152: mol_type = other DNA
gaattcgttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctgggtaccaggcgcgcatttcattttgtttttttctatgctataaatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggcggcccgctcgagaaaaattgaaaataaatacaaaggttcttgagggttgtgttaaattgaaagcgagaaataatcataaataagccaccaccgtttaaacagtcgacggtatcgataagcttgatatcgaattcctgcagcccgtacgcgcaggcagcatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttgatttttatggcgcgccctgcagggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccagaaactagttaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 31 - DNA, 2709 bp, Synthetic construct
  source 1..2709: mol_type = other DNA
atggtcaaacagattaaggttcgagtggacatggtgcggcatagaatcaaggagcacatgctgaaaaaatatacccagacggaagagaaattcactggcgcctttaatatgatgggaggatgtttgcagaatgccttagatatcttagataaggttcatgagcctttcgaggagatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcactgcagaacttgcctcagtgctcccctgatgagattatggcttatgcccagaaaatatttaagattttggatgaggagagagacaaggtgctcacgcacattgatcacatatttatggatatcctcactacatgtgtggaaacaatgtgtaatgagtacaaggtcactagtgacgcttgtatgatgaccatgtacggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctctgagagtctgctctcctagtgtggatgacctacgggccatcgccgaggagtcagatgaggaagaggctattgtagcctacactttggccaccgctggtgtcagctcctctgattctctggtgtcacccccagagtcccctgtacccgcgactatccctctgtcctcagtaattgtggctgagaacagtgatcaggaagaaagtgagcagagtgatgaggaagaggaggagggtgctcaggaggagcgggaggacactgtgtctgtcaagtctgagccagtgtctgagatagaggaagttgccccagaggaagaggaggatggtgctgaggaacccaccgcctctggaggcaagagcacccaccctatggtgactagaagcaaggctgaccagggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtgcgcaatatcatgaaagataagaacacccccttctgcacacccaacgtgcagactcggcggggtccgcgtcaagattgacgaggtgagccgcatgttccgcaacaccaatcgctctcttgagtacaagaacctgcccttcacgattcccagtatgcaccaggtgttagatgaggccatcaaagcctgcaaaaccatgcaggtgaacaacaagggcatccagattatctacacccgcaatcatgaggtgaagagtgaggtggatgcggtgcggtgtcgcctgggcaccatgtgcaacctggccctctccactcccttcctcatggagcacaccatgcccgtgacacatccacccgaagtggcgcagcgaacagccgatgcttgtaacgaaggcgtcaaggccgcgtggagcctcaaagaattgcacacccaccaattatgcccccgttcctccgattaccgcaacatgatcatccacgctgccacccccgtggacctgttgggcgctctcaacctgtgcctgcccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcagtaa